(19) United States
(12) Patent Application Publication
(10) Pub. No.: US 2024/0249859 A1
(43) Pub. Date: Jul. 25, 2024

(54) A CABLE ASSEMBLY FOR AN ELECTROSURGICAL INSTRUMENT, AND A METHOD FOR MANUFACTURING THE SAME

(71) Applicant: Creo Medical Limited, Chepstow, Monmouthshire (GB)

(72) Inventors: Christopher Paul Hancock, Chepstow, Monmouthshire (GB); Malcolm White, Chepstow, Monmouthshire (GB)

(73) Assignee: Creo Medical Limited, Chepstow, Monmouthshire (GB)

(21) Appl. No.: 18/577,224

(22) PCT Filed: Jun. 27, 2022
(86) PCT No.: PCT/EP2022/067601

§ 371 Date: Jan. 5, 2024

(30) Foreign Application Priority Data
Jul. 6, 2021 (GB) 2109754.8

(51) Int. Cl. H01B 9/04; A61B 18/00; A61B 18/12; A61B 18/18; A61B 90/30; H01B 9/00; H01B 13/016

(52) U.S. Cl. CPC H01B 9/04; A61B 18/12; A61B 18/1815; A61B 90/30; H01B 9/005; H01B 13/016; A61B 2018/00178; A61B 2090/306

(57) ABSTRACT

The invention relates to a cable assembly for an electrosurgical instrument, comprising an inner conductive layer, an outer conductive layer arranged coaxially with the inner conductive layer, a dielectric layer separating the inner conductive layer and the outer conductive layer, and an optical fibre for transmitting electromagnetic radiation in the ultraviolet spectrum, the visible spectrum, and/or in the infrared spectrum; wherein the inner conductive layer, the dielectric layer, and the outer conductive layer form a coaxial cable providing a transmission line for conveying radiofrequency and/or microwave radiation, wherein the inner conductive layer surrounds the optical fibre, and wherein the inner conductive layer and the optical fibre are bonded to each other.

### TECHNICAL FIELD

The present invention relates to a cable assembly for an electrosurgical instrument, comprising an inner conductive layer, an outer conductive layer arranged coaxially with the inner conductive layer, a dielectric layer separating the inner conductive layer and the outer conductive layer, and an optical fibre configured to transmit electromagnetic radiation in the ultraviolet spectrum, the visible spectrum, and/or in the infrared spectrum.

The invention also relates to a method for manufacturing a cable assembly.

### BACKGROUND OF THE INVENTION

Electrosurgical instruments are instruments that are used to deliver radiofrequency and/or microwave frequency energy to biological tissue, for purposes such as cutting biological tissue or coagulating blood. Radiofrequency and/or microwave frequency energy is supplied to the electrosurgical instrument using a cable. Conventional cables used for this purpose have a coaxial transmission line structure comprising a solid cylindrical inner conductor, a tubular layer of dielectric material around the inner conductor, and a tubular outer conductor around the dielectric material.

When operating many electrosurgical instruments, it is common to need to provide additional supplies or components (e.g. control means) to the electrosurgical instrument, such as a liquid or gas feed, liquids or gasses, or guide- or pull-wires for manipulating (for example opening/closing, rotating, or extending/retracting) part(s) of the electrosurgical instrument.

In order to provide these additional supplies or components to the electrosurgical instrument, additional structures have been provided together with the conventional cable, such as additional tubes adjacent to the conventional cable. For example, it is known to provide an additional tube housing a pull-wire for the electrosurgical instrument alongside the conventional cable, and to house the conventional cable and the tube housing the pull-wire within a single protective jacket/casing.

### SUMMARY OF THE INVENTION

An objective of the invention is to provide a cable assembly configured to convey radiofrequency and/or microwave radiation as well as electromagnetic radiation in the ultraviolet spectrum, the visible spectrum, and/or in the infrared spectrum and having reduced dimensions.

The subject matter of the independent claims has been developed in light of this objective. The dependent claims describe optional embodiments of the invention.

A cable assembly for an electrosurgical instrument comprises an inner conductive layer, an outer conductive layer arranged coaxially with the inner conductive layer, a dielectric layer separating the inner conductive layer and the outer conductive layer, and an optical fibre configured to transmit electromagnetic radiation in the ultraviolet spectrum, the visible spectrum, and/or in the infrared spectrum. The inner conductive layer, the dielectric layer, and the outer conductive layer form a coaxial cable providing a transmission line for conveying radiofrequency and/or microwave radiation. The inner conductive layer surrounds the optical fibre. The inner conductive layer and the optical fibre are bonded to each other.

An electrosurgical instrument may be any instrument, or tool, which is used during surgery and which utilises radiofrequency and/or microwave frequency energy. Herein, radiofrequency (RF) may mean a stable fixed frequency in the range 10 kHz to 300 MHz and microwave radiation may mean electromagnetic radiation having a stable fixed frequency in the range 300 MHz to 100 GHz. The RF radiation may have a frequency high enough to prevent the energy from causing nerve stimulation and low enough to prevent the energy from causing tissue blanching or unnecessary thermal margin or damage to the tissue structure. Preferred spot frequencies for the RF energy include any one or more of: 100 kHz, 250 kHz, 400 kHz, 500 KHz, 1 MHz, or 5 MHz. Preferred spot frequencies for the microwave energy include 915 MHZ, 2.45 GHZ, 5.8 GHZ, 14.5 GHZ, 24 GHZ.

The cable assembly may also be configured for conveying direct current (DC) using the inner conductive layer and/or the outer conductive layer. In other words, the coaxial cable of the cable assembly may be used to transmit electrical signals or electrical energy from direct current up to microwave frequencies, while the optical fibre of the cable assembly permits delivery of possibly ultraviolet (UV), optical and infrared (IR) energy.

Infrared is understood to encompass wavelengths from the nominal red edge of the visible spectrum around 700 nm (frequency 430 THz), to 1 mm (300 GHz). The visible spectrum is understood to encompass wavelengths from about 380 nm (789 THz) to about 750 nm (400 THz). Ultraviolet (UV) is understood to encompass wavelengths from 10 nm (with a corresponding frequency around 30 PHz) to 400 nm (750 THz). One example of infrared (IR) is 193 THz, or 1550 nm.

The electrosurgical instrument may be an endoscopic instrument, i.e. an instrument that can be inserted into a cavity of a body for treating tissue close to or in the cavity of the body. For example, the electrosurgical instrument is configured for ablating, coagulating, cutting and/or resecting tissue at, within, or close to the cavity of the body. The electrosurgical instrument may be suitable for use with an endoscope or another type of scoping device (e.g. a bronchoscope).

A first end of the cable assembly is the end of the cable that is for connecting (either directly or indirectly through another component or part) to the electrosurgical instrument. In other words, the first end of the cable assembly is the distal end of the cable.

An opposite, second end of the cable assembly is for connecting the cable to the generator for supplying radiofrequency and/or microwave frequency energy to the cable and to a light source for generating electromagnetic radiation in the ultraviolet spectrum, the visible spectrum, and/or in the infrared spectrum. In other words, the second end of the cable assembly is the proximal end of the cable. The second end of the cable assembly may have a terminal or connector for connecting the second end of the cable to the generator and/or light source. Thus, the cable assembly may be for conveying radiofrequency and/or microwave frequency energy from a generator connected to the second (proximal) end of the cable assembly to an electrosurgical instrument connected to the first (distal) end of the cable. In addition, the cable assembly may be for conveying ultraviolet, visible, or infrared light from the light source connected to the second (proximal) and of the cable assembly to the electrosurgical instrument connected to the first (distal) end of the cable assembly.

The light source may include one or more elements for generating light of a single frequency or of a plurality of frequencies. For example, the light source may include a laser, a laser diode, a LED, and/or a white light source. The light generated by the light source may be used for illuminating, cutting, coagulating, treating and/or analysing tissue within or at the cavity. For example, laser light may be used for cutting tissue and/or for Raman spectroscopy.

The inner conductive layer may completely surround the optical fibre. However, it is possible that the inner conductive layer partially surrounds the optical fibre. It is solely important that the inner conductive layer and the outer conductive layer are coaxially arranged in order to form a coaxial cable capable of transmitting microwave and/or radiofrequency radiation. To this end, the outer conductive layer may completely or partially surround the inner conductive layer. In particular, an angular degree of extension of the inner conductive layer along a circumferential direction corresponds to an angular degree extension of the outer conductive layer along the circumferential direction. That is, the inner conductive layer may be radially aligned with, or may radially overlap with, the outer conductive layer. The inner conductive layer and the outer conductive layer may not form a complete circle in a cross-sectional view of the coaxial cable. For example, the inner conductive layer and/or the outer conductive layer extend only to 3/4, 4/5, or other ratios of the complete circumference of the coaxial layer.

Optionally, the inner conductive layer, the dielectric layer, and/or the outer conductive layer have a ring shape in a cross-sectional view of the cable assembly. In particular, the inner conductive layer, the dielectric layer, and/or the outer conductive layer have a circular shape in the cross-sectional view of the cable assembly. Thus, the inner conductive layer is not a solid body but is hollow to define a cavity that extends along an axial direction of the cable assembly. The optical fibre is positioned in this cavity.

The inner conductive layer and/or the outer conductive layer are made from an electrically conductive material, such as metal. The inner conductive layer can be made from copper, aluminium, and/or tin.

The dielectric layer is provided for electrically insulating the inner conductive layer from the outer conductive layer. The dielectric layer may be made from an electrically insulating material. The dielectric layer may also have a ring shape in a cross-sectional view of the cable assembly, in particular a circular shape. The inner conductive layer, the outer conductive layer, and/or dielectric layer can be coaxially arranged to each other and each may have a tubular shape.

The coaxial cable, in particular the outer conductive layer, is covered or wrapped in a cover layer which is provided for mechanical strength, proofing against entry of chemicals, such as water, and/or shielding the coaxial cable from mechanical damage. The cover layer may be made from a plastic material and can be electrically insulating.

The inner conductive layer, the dielectric layer, the outer conductive layer, the cover layer, and/or the optical fibre can continuously extend from the distal end to the proximal end of the cable assembly.

The optical fibre is a fibre for conveying electromagnetic radiation by reflection, for example, total internal reflection. The optical fibre may be configured to convey a single mode radiation or multimode radiation. The optical fibre can be used as a means to transmit light between the two ends of the cable assembly. An optical fibre generally includes a core (or core portion) surrounded by a transparent cladding material (or cladding layer) with a lower index of refraction. Light is kept in the core by reflection (e.g. total internal reflection) which causes the fibre to act as a waveguide.

The inner conductive layer is permanently attached to the optical fibre by bonding the inner conductive layer to the optical fibre. In other words, the inner conductive layer is permanently fixed to the optical fibre. For example, the inner conductive layer cannot be removed from the optical fibre without destroying the inner conductive layer and/or the optical fibre. Adhesive bonding and/or adhesive agents may be used for bonding the inner conductive layer to the optical fibre. However, it is also possible that the inner conductive layer is directly bonded to the optical fibre (e.g. without an adhesive agent in-between).

The inner conductive layer and the optical fibre may form a unitary component. In particular, the inner conductive layer and the optical fibre may be constituted by a coated optical fibre. This means, the inner conductive layer and the optical fibre forming a coated optical fibre may be a pre-assembled component that may be purchased as it is.

Coated optical fibres are often used in harsh environments; for example when the optical fibre is subjected to high temperature environments, harsh chemical environments, ionising radiation and/or other influences which may attack the integrity of the optical fibre. For example, coated optical fibres may have a working temperature range from −270° ° C. to 600° C. and up to a humidity of up to 100%.

Conventionally, the coating applied to such known coated optical fibres is used to protect from damage the more delicate optical fibre beneath the coating. However, according to various embodiments, the optical fibres are coated with a metal coating or cover which acts as the inner conductive layer for the cable assembly.

The cable assembly may be manufactured by covering or wrapping the coated optical fibre with the dielectric layer. Afterwards, the dielectric layer is covered or wrapped with the outer conductive layer. Thus, the cable assembly may be manufactured based on the prefabricated coated optical fibre which constitutes the optical fibre and the inner conductive layer.

The cable assembly as well as a method for manufacturing the cable assembly provide a simple way for manufacturing a cable assembly which includes a transmission line for microwave or radiofrequency radiation and for radiation in the ultraviolet, visible and/or infrared spectrum. Since coated optical fibres can be manufactured with small outer diameters, the cable assembly may also have a small outer diameter which is helpful if the cable assembly is used in conjunction with an electrosurgical instrument that is an endoscopic instrument, i.e. an instrument that is inserted into a cavity of a body to be treated. Metal coated optical fibres are available at around 0.35 mm diameter.

Rather surprisingly, the bending radius of the coated optical fibre is similar to the bending radius of known coaxial cables. Thus, using a pre-assembled coated optical fibre results in a cable assembly with bending characteristics similar to existing coaxial cables.

At its most general, the present invention proposes to make a cable assembly using wrapping technology which is known for use in the manufacture of the known coaxial cables, but with the centre-conductor replaced by a coated optical fibre.

In an optional embodiment, the inner conductive layer and the optical fibre are bonded to each other by a chemical bond, further optionally by a molecular bond.

As understood herein, a chemical bond is a lasting attraction between atoms, ions or molecules that enables the formation of chemical compounds. The bond may result from electrostatic forces of attraction between oppositely charged ions as in ionic bonds or through the sharing of electrons as in covalent bonds. The strength of chemical bonds varies considerably; there are “strong bonds” or “primary bonds” such as covalent, ionic, and metallic bonds, and “weak bonds” or “secondary bonds” such as dipole-dipole interactions, the London dispersion force and hydrogen bonding. Furthermore, as understood herein, a covalent bond is a chemical bond that involves the sharing of electron pairs between atoms. These electron pairs are known as shared pairs or bonding pairs, and the stable balance of attractive and repulsive forces between atoms, when they share electrons, is known as covalent bonding.

Thus, the optical fibre is not fixed to the inner conductive layer by friction or other mechanical means, but by forming a chemical bond between the optical fibre and the inner conductive layer.

All means to bond the metal inner conductive layer to the silicon based optical fibre are possible. For example, a bonding between the inner conductive layer and the optical fibre may be achieved by sufficiently heating the inner conductive layer when applying the inner conductive layer to the optical fibre.

After the conductive layer has been added to the optical fibre, the conductive layer can be soldered or brazed to other metals, i.e. the bond between the optical fibre and the inner conductive layer is robust and able to withstand these subsequent processes.

In an optional embodiment, the optical fibre includes a core portion and a cladding layer which surrounds the core portion, wherein optionally the inner conductive layer and the cladding layer bonded to each other.

The core portion is that part of the optical fibre in which the electromagnetic radiation propagates. The cladding layer usually has a lower refractive index compared to the core portion such that total internal reflection occurs at the interface between the core portion and the cladding layer.

The bonding of the cladding layer to the core portion may be achieved during coextrusion of the core portion and the cladding layer. However, all means known in the prior art for attaching the cladding layer to the core portion can be used.

The core portion may include (pure) Silica (SiO₂) while the cladding layer can include doped silica, for example Fluorine doped Silica (F:SiO₂). However, other materials commonly used with optical fibres can be used.

In this embodiment, the inner conductive layer is bonded to the cladding layer, optionally by a chemical or covalent bond. The cladding layer completely surrounds the core portion in a cross-sectional view of the optical fibre. What is more, the inner conductive layer also completely surrounds the optical fibre in a cross-sectional view of the optical fibre.

In an optional embodiment, the core portion has a diameter between 6 μm and 650 μm, the cladding layer has a diameter between 80 μm and 700 μm, and/or the inner conductive layer has a diameter between 120 μm and 800 μm, optionally 350 μm.

The diameter refers to the largest distance of the respective component in a cross-sectional view (e.g. the outer or outermost diameter). The optical fibre optionally has a circular cross-section such that the diameter corresponds to the mathematical diameter of a circle.

The diameter of the inner conductive layer corresponds to the outer diameter of the coated optical fibre. The cladding layer may have a thickness corresponding to 5% to 20% of the diameter of the core portion.

In an optional embodiment, the cable assembly further comprises a coating layer surrounding the inner conductive layer.

The coating layer is provided for protecting the inner conductive layer, for example from oxidation (e.g. corrosion) or other chemical reactions of the inner conductive layer with its surroundings. In particular, the coating layer protects an outer surface of the inner conductive layer.

The coating layer may be applied to the pre-assembled coated optical fibre. To this end, the outer conductive layer is covered or wrapped with the coating layer. The coating layer may include a conductive material, such as a metal, and may be bonded to the inner conductive layer. However, any other known techniques for applying a conductive layer on a metal layer can be used.

In an optional embodiment, the coating layer includes silver or gold, wherein optionally the silver coating layer has a thickness corresponding to (e.g. substantially equal to or greater than) the Skin depth of the radiofrequency and/or microwave radiation or wherein the gold coating layer has a thickness smaller than the Skin depth of the radiofrequency and/or microwave radiation. For example, the thickness of the coating layer may be equal to the Skin depth 20%, 10%, or 5% of the Skin depth.

The silver coating layer not only protects the metal inner conductive layer especially if the inner conductive layer is made from copper, but also contributes to the transmission of the radiofrequency or microwave current in the inner conductive layer and the coating layer. This is because the thickness of the silver coating layer is equal or larger than the Skin depth within which most of the electromagnetic current propagates. For example, the thickness of the silver coating layer is between 5 μm and 20 μm, optionally 10 μm.

A gold coating layer may have a reduced thickness compared to the silver coating layer if the inner conductive layer has a higher electrical conductivity than gold, for instance if it is copper. Gold is chemically inert such that it reliably protects the inner conductive layer.

In an optional embodiment, the cable assembly further comprises a combiner including a common transmission line section, a first transmission line section, and a second transmission line section. Optionally, an input of the common transmission line section is in communication with outputs of the first transmission line section and the second transmission line section.

The combiner may be attached to the proximal end of the cable. Thus, the cable assembly includes the coaxial cable, the optical fibre, and the combiner. The combiner may be configured as a diplexer. The combiner is provided for combining the radiofrequency and/or microwave radiation generated by the generator with the electromagnetic radiation generated by the light source. In other words, the combiner combines the input into the optical fibre with the input into the coaxial cable.

The common transmission line section, the first transmission line section, and the second transmission line section may form a junction or T-junction. For example, the common transmission line section and the first transmission line section may extend in a line from which the second transmission line section protrudes or splits off (e.g. at a substantially 90 degree angle).

The common transmission line section may be connected to (or may include) the coaxial cable and the optical fibre as described before, the first transmission line section may be in communication with (e.g. directly and/or indirectly connected to or coupled to) the light source, and the second transmission line section may be in communication with (e.g. directly and/or indirectly connected to or coupled to) the generator.

Thus, the first transmission line section may be configured to solely convey electromagnetic radiation generated by the light source whereas the second transmission line section may be configured to solely convey radiofrequency and/or microwave radiation generated by the generator. The common transmission line section (which may include the coaxial cable and the optical fibre) may be configured to convey both types of radiation.

In an optional embodiment, the common transmission line section includes a common optical fibre section connected to (e.g. including) the optical fibre, a common inner conductor connected to (e.g. including) the inner conductive layer, and/or a common outer conductor connected to (e.g. including) the outer conductive layer. Optionally, the first transmission line section includes a first optical fibre section in communication with (e.g. directly and/or indirectly connected to or coupled to) the common optical fibre section (e.g. at a distal end) and configured to be connected to the light source (e.g. at a proximal end), and/or a first outer conductor in communication with (e.g. directly and/or indirectly connected to or coupled to) the common outer conductor (e.g. at a distal end). Further optionally, the second transmission line section includes a second inner conductor in communication with (e.g. directly and/or indirectly connected to or coupled to) the common inner conductor (e.g. at a distal end) and configured to be connected to an external radiofrequency or microwave energy source or generator (e.g. at a proximal end), a second dielectric portion electrically insulating the second inner conductor, and/or a second outer conductor in communication with (e.g. directly and/or indirectly connected to or coupled to) the common outer conductor (e.g. at a distal end).

In an optional embodiment, the common dielectric portion, first dielectric portion, and the second dielectric portion electrically insulate the common inner conductor, the first inner conductor, and the second inner conductor, respectively, from an electrically conductive body, which is described below in more detail. In this case, the common outer conductor, the first outer conductor, and/or the second outer conductor can be omitted, for example, because the functionality thereof is provided by the electrically conductive body.

The common transmission line section may have features and characteristics similar to the coaxial cable and/or optical fibre. For example, the common inner conductor may be bonded to the common optical fibre section in a similar way as the inner conductive layer is bonded to the optical fibre. The common inner conductor and the common outer conductor may form a coaxial cable similar to the inner conductive layer and the outer conductive layer, respectively.

The first transmission line section and/or the second transmission line section may deviate from the configuration of the common transmission line section as will be explained in the following.

In an optional embodiment, the combiner includes an electrically conductive body, wherein the combiner is devoid of the common outer conductor, the first outer conductor and/or the second outer conductor.

The electrically conductive body may be a cuboid having three holes in which the common transmission line section, the first transmission line section and the second transmission line section extend. The three holes may intersect at one point forming the junction or T-junction. The electrically conductive body may be a solid block made from a metal material in which the above-mentioned holes may be drilled.

The electrically conductive body may be grounded and in contact with the common outer conductor and/or the first outer conductor for grounding these conductors. Thus, the common outer conductor and/or the first outer conductor are not covered by an electrically insulating cover or wrapping (cover layer) as the coaxial cable can be.

The second transmission line section may also include an outer conductor which is in contact with the electrically conductive body. However, the electrically conductive body may act as the outer conductor for the common transmission line section, the first transmission line section, and/or the second transmission line section.

The body of the combiner may be made from an electrically non-conductive material, such as plastic material. The electrically non-conductive material requires the presence of the common outer conductor, the first outer conductor and the second outer conductor.

In an optional embodiment, the first transmission line section includes a first inner conductor in communication with (e.g. directly and/or indirectly connected to or coupled to) the common inner conductor, and a length of the first inner conductor between an end of the first inner conductor and a junction of the second transmission line section and the common transmission line section is equal to n*λ/2, wherein n is an integer equal to or greater than 0 and λ is the wavelength of the conveyed radiofrequency and/or microwave radiation. Further optionally, the first transmission line section includes an additional first inner conductor which is arranged axially spaced apart from the end of the first inner conductor. That is, when present, the additional first inner conductor is spaced from the first inner conductor by a gap.

In other words, the first inner conductor and/or the additional first inner conductor do not fully extend along the extension of the first transmission line section—only the optical fibre section and the first outer conductor fully extend along the extension of the first transmission line section. This gap is provided for ensuring that frequencies below 100 GHz are stopped from being conveyed along the first transmission line section, in particular the first inner conductor.

The position where the second transmission line section is in contact with (e.g. joins with or forms a junction with) the common transmission line section may correspond to the aforementioned junction or T-junction. Thus, in an optional embodiment, the first inner conductor ends at a T-junction or at n*λ/2 after the T-junction. These distances between the T-junction and the end of the first inner conductor reduce or eliminate back reflection of the radiofrequency and/or microwave radiation at the end of the first inner conductor.

The additional first inner conductor may have the same characteristics, features, and/or optional embodiments as the first inner conductor. The first inner conductor and the additional first inner conductor are not in electrical contact with each other but spaced apart by a gap.

In other words, the first inner conductor may terminate at the above defined end and the additional first inner conductor is arranged spaced apart from the first inner conductor such that there is a gap between the first inner conductor and the additional first inner conductor. This gap is of sufficient length such that no electromagnetic coupling is possible over the gap.

The provision of the gap simplifies the manufacturing of the first transmission line section since the coated optical fibre can also be used for the first transmission line section and/or the additional first inner conductor. It is only necessary to remove the inner conductive layer (or the coating of the coated optical fibre) over the extension of the gap. This creates the first inner conductor and/or the additional first inner conductor. The position of the gap is appropriately chosen such that the above-described definitions apply.

In an alternative optional embodiment, the first transmission line section includes no first inner conductor and/or no additional first inner conductor. In other words, there is no first inner conductor and/or no additional first inner conductor over the complete extension of the first transmission line section.

This embodiment corresponds to the end of the first inner conductor at the T-junction (or n=0) and there is no additional first inner conductor. This embodiment facilitates the suppression of propagation of radiofrequency and/or microwave radiation along the first transmission line section over a broad bandwidth of frequencies of radiation.

The transmission line section of this embodiment may be manufactured without using a coated optical fibre but instead by using an uncoated optical fibre and covering this uncoded optical fibre with a first dielectric portion and/or the first outer conductor.

In an optional embodiment, the common transmission line section includes a common dielectric portion arranged between the common inner conductor and the common outer conductor, and the first transmission line section includes a first dielectric portion arranged between the first optical fibre section and the first outer conductor. Further optionally, the common dielectric portion, the first dielectric portion, and/or the second dielectric portion include solid polytetrafluoroethylene (PTFE), wherein optionally the dielectric layer includes expanded PTFE.

The dielectric layer of the coaxial cable may be coextruded onto the coated optical fibre. Since the common dielectric portion, the first dielectric portion, and/or the second dielectric portion are arranged within the combiner, coextrusion of these dielectric portions may not be possible such that a solid PTFE is used instead. The common dielectric portion, the first dielectric portion, and/or the second dielectric portion may have a tubular shape. The common dielectric portion and the first dielectric portion may be connected to each other or form a single component.

The second dielectric portion may electrically insulate the second inner conductor from the electrically conductive body or the optional second outer conductor.

The second transmission line section may not include an optical fibre such that the second inner conductor may not include a cavity as the common inner conductor and/or the first inner conductor. The second inner conductor may be a solid conductor. The second inner conductor may be electrically connected to an outer surface of the common inner conductor.

In an optional embodiment, the combiner includes at least one choke for preventing propagation of radiofrequency and/or microwave radiation, wherein the at least one choke is connected to the first transmission line section.

One or more filters or chokes are provided for stopping the radiofrequency and/or microwave radiation from continuing on the outside of the first transmission line section (i.e. on the first inner and/or outer conductor), and for facilitating that they pass smoothly from the second transmission line section to the distal end of the coaxial cable. The choke may act as a band-stop filter whose bandwidth depends on its configuration. The smooth transition of radiofrequency and/or microwave radiation from the second transmission line section to the distal end of the coaxial cable depends on the position along the first transmission line section. For example, each choke may be positioned at n*λ/2 from the T-junction, wherein n is an integer equal to or greater than 0 and λ is the wavelength of the conveyed radiofrequency and/or microwave radiation in the first transmission line system. Providing multiple chokes, wherein each choke is positioned to suppress a different wavelength, means that multiple wavelengths can be suppressed from propagating along the first transmission line section. This may be achieved by connecting a microwave combiner and/or a microwave/RF combiner to the second transmission line section such that radiation of multiple frequencies is conveyed by the second transmission line section, the common transmission line section, and/or the coaxial cable.

In an optional embodiment, the choke includes a cavity within the electrically conductive body or the electrically non-conductive body which is in fluid communication (e.g. fluid connection) with the first outer conductor and/or the first dielectric portion, wherein optionally in a cross-sectional view of the choke, a half of the choke has an L shape.

The choke may be symmetrical around the first transmission line section. Thus, the choke may have an overall U-shaped configuration in a cross-sectional view, wherein the first transmission line section intersects with the base of U-shaped configuration. The base of the U-shaped configuration in a cross-sectional view may extend perpendicular to the first transmission line section while the arms of U-shaped configuration may extend parallel to the first transmission line section. In other words, the choke may have an overall beaker-shaped configuration wherein the bottom of this beaker intersects with the first transmission line section. The beaker is axially symmetrical to the first transmission line section.

The choke may be a cavity within the (electrically conductive) body of the combiner having a constant thickness and the above-described configuration. Thus, the first outer conductor is not completely in contact with the body, namely for the section at which the choke is in fluid communication with the first outer conductor.

A method for manufacturing a cable assembly comprises the steps of a) providing a coated optical fibre, wherein the coated optical fibre includes an optical fibre which is coated by a conductive layer and wherein the conductive layer is bonded to the optical fibre, b) covering or wrapping the coated optical fibre with a dielectric layer, and c) covering or wrapping dielectric layer with an outer conductive layer.

The features, characteristics and optional embodiments of the cable assembly described above equally apply to the method for manufacturing the cable assembly. The conductive layer may correspond to the above-described inner conductive layer.

In an optional embodiment, the coated optical fibre is coated with a coating layer before step b), wherein the coating layer includes silver or gold.

### BRIEF DESCRIPTION OF THE DRAWINGS

For a better understanding of the invention, and to show how embodiments of the same may be carried into effect, reference will now be made, by way of example, to the accompanying drawings, in which:

FIG. 1 shows a cross-sectional view of a coaxial cable and an optical fibre of a cable assembly;

FIG. 2 shows a cross-sectional view of a combiner of the cable assembly;

FIG. 3 shows a cross-sectional view of a further embodiment of a combiner;

FIG. 4 shows a cross-sectional view of a further embodiment of a combiner; and

FIG. 5 shows a block diagram outlining a method for manufacturing a cable assembly.

### DETAILED DESCRIPTION; FURTHER OPTIONS AND PREFERENCES

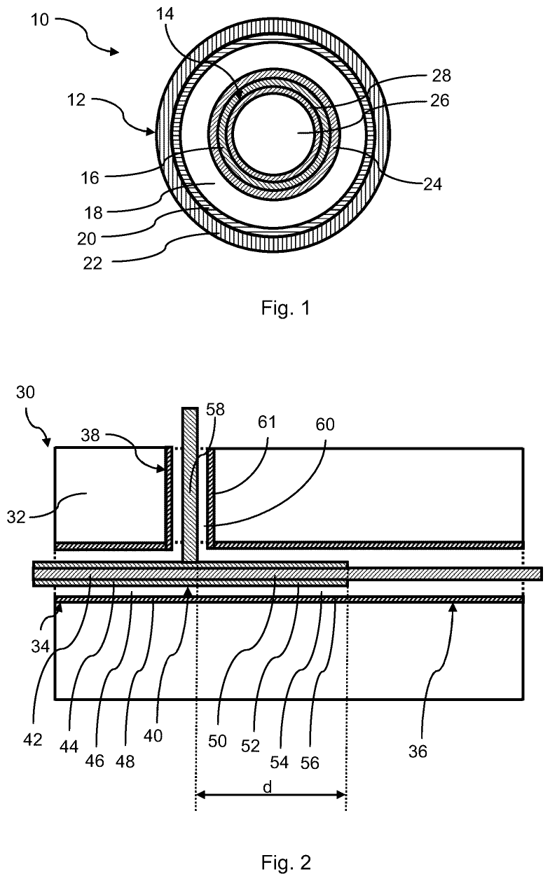

With reference to FIG. 1, a cable assembly 10 includes a coaxial cable 12 and an optical fibre 14. The coaxial cable 12 includes an inner conductive layer 16, a dielectric layer 18, an outer conductive layer 20, and/or a cover layer 22. The inner conductive layer 16 is electrically insulated from the outer conductive layer 20 by the dielectric layer 18. The inner conductive layer 16 and the outer conductive layer 20 form the coaxial cable 12 configured for conveying radiofrequency and/or microwave frequency radiation.

The inner conductive layer 16 and the outer conductive layer 20 are made from electrically conductive materials, such as metals. The dielectric layer 18 may be made from expanded PTFE. The cover layer 22 is made from an electrically insulating material, such as a plastic material. The cover layer 22 is provided for covering or shielding the coaxial cable 12 from external influences.

The inner conductive layer 16 has a ring shape in a cross-sectional view of the coaxial cable 12 and can be made from copper. A cavity that is provided by the inner conductive layer 16 is filled by the optical fibre 14. In other words, the coaxial cable 12, in particular the inner conductive layer 16, surrounds the optical fibre 14. The optical fibre 14 is configured to convey electromagnetic radiation in the ultraviolet spectrum, visible spectrum, and/or in the infrared spectrum.

The optical fibre 14 is bonded to the inner conductive layer 16, for example by a chemical, in particular a molecular bond. Adhesive agents may be used for permanently fixing the inner conductive layer 16 to the optical fibre 14. The optical fibre 14 and the inner conductive layer 16 may be a pre-assembled coated optical fibre.

The coaxial cable 12 may further include a coating layer 24. The coating layer 24 can be made from an electrically conductive material and is arranged on the inner conductive layer 16, for example on an outer surface of the inner conductive layer 16. Thus, the coating layer 24 is arranged between the inner conductive layer 16 and the dielectric layer 18. The coating layer 24 is provided for shielding the inner conductive layer 16 from external influences. For example, the coating layer 24 reduces oxidation, such as corrosion, or other chemical effects on the inner conductive layer 16, or improves electrical conductivity.

The coating layer 24 may be made from gold or silver. The thickness of a silver coating layer 24 may roughly correspond to several skin depths of the electromagnetic radiation propagating within the coaxial cable 12. The thickness of the silver coating layer 24 may be approximately 5 μm. Thus, the electric current that would have flowed within the inner conductive layer 16 may mainly be conducted in the silver coating layer 24. This reduces the loss of the coaxial cable 12.

The thickness of a gold coating layer 24 may be less than the Skin depth such that the electric current flowing within the inner conductive layer 16 mainly propagates within the inner conductive layer 16. Gold is chemically inert such that it reliably protects the inner conductive layer 16.

The optical fibre 14 includes a core portion 26 and/or a cladding layer 28. The core portion 26 and the cladding layer 28 are shaped and configured to convey the electromagnetic radiation. A refractive index of the cladding layer 28 is lower than a refractive index of the core portion 26 such that electromagnetic radiation propagating in the core portion 26 is reflected at an interface between the core portion 26 and the cladding layer 28 by total internal reflection. Thus, the electromagnetic radiation conveyed by the optical fibre 14 effectively propagates solely within the core portion 26. The cladding layer 28 may be proportionally thicker than the core portion 26 than is shown in the attached drawings.

The core portion 26 and the cladding layer 28 can be made from dielectric materials. For example, the core portion 26 is made from silica and the cladding layer 28 is made from fluorine doped silica.

The inner conductive layer 16 may be bonded to the cladding layer 28. The core portion 26, the cladding layer 28, and the inner conductive layer 16 may form a prefabricated coated optical fibre.

The cable assembly 10 may further comprise a combiner 30 which is connected to a proximal end of the coaxial cable 12 and the optical fibre 14. The combiner 30 is provided for combining the output of a generator (not shown in the figures) generating the radiofrequency and/or microwave radiation with the output of a light source (not shown in the figures) generating the electromagnetic radiation in the ultraviolet, visible, and/or infrared spectrum and for inputting it into the coaxial cable 12 and the optical fibre 14, respectively.

The combiner 30 includes a body 32, a common transmission line section 34, a first transmission line section 36, and a second transmission line section 38. The outputs of the first transmission line section 36 and the second transmission line section 38 are input in the common transmission line section 34 forming a T-junction 40.

The common transmission line section 34 is connected to the coaxial cable 12 and the optical fibre 14. The first transmission line section 36 and the second transmission line section 38 are connected to the light source and the generator, respectively.

The common transmission line section 34 includes a common optical fibre section 42, a common inner conductor 44, a common dielectric portion 46, and/or a common outer conductor 48. The first transmission line section 36 includes a first optical fibre section 50, a first inner conductor 52, a first dielectric portion 54, and/or a first outer conductor 56. The second transmission line section 38 includes a second inner conductor 58, a second dielectric portion 60, and/or a second outer conductor 61.

The common optical fibre section 42 is connected to the optical fibre 14 and the first optical fibre section 50. The common inner conductor 44 is connected to the inner conductive layer 16, the first inner conductor 52, and the second inner conductor 58. The common dielectric portion 46, the first dielectric portion 54, and the second dielectric portion 60 may be separate components but in direct contact with each other. These dielectric portions 46, 54, and 60 may be made from solid materials such as PTFE. The common outer conductor 48 is connected to the first outer conductor 56 and the second outer conductor 61.

The body 32 is made from an electrically non-conductive material such as plastic. The body 32 may be a solid block into which three holes are drilled intersecting at the T-junction 40. The common transmission line section 34, the first transmission line section 36, and the second transmission line section 38 are each inserted in one of the holes drilled into the body 32.

If the body 32 of the combiner 30 is made from a non-conductive material, the common outer conductor 48, the first outer conductor 56, and the second outer conductor 61 need to be present for providing coaxial transmission lines.

In another embodiment, the body 32 is made from an electrically conductive material such as metal. The common outer conductor 48, the first outer conductor 56, and the second outer conductor 61 may be in electrical contact with the body 32 which can be electrically grounded. If the body 32 is made from an electrically conductive material, the common outer conductor 48, the first outer conductor 56, and the second outer conductor 61 can be omitted since the electrically conductive body 32 acts as the common outer conductor 48, the first outer conductor 56, and the second outer conductor 61.

The first inner conductor 52 may extend from the T-junction 40 over a distance d. Thus, the first inner conductor 52 does not extend over the complete length of the first transmission line section 36. This is done in order to stop electromagnetic radiation in the radiofrequency spectrum or microwave spectrum, in particular up to 100 GHZ, from propagating along the first transmission line section 36.

In order to reduce backscattering at the end of the first inner conductor 52, the distance d is chosen to be n*λ/2, wherein n is an integer equal to or greater than 0 and λ is the wavelength of the conveyed radiofrequency and/or microwave radiation in the first transmission line section 36.

The second transmission line section 38 does not include an optical fibre. This allows that the second inner conductor 58 may not include a cavity as with the common inner conductor 44 or the inner conductive layer 16. Instead, the second inner conductor 58 may be solid.

The common optical fibre section 42 and the first optical fibre section 50 may form a unitary optical fibre. Thus, the common optical fibre section 42 and the first optical fibre section 50 may be manufactured by placing a single optical fibre in the holes drilled into the body 32. Alternatively, a single coated optical fibre may be placed in the holes drilled into the body 32. Parts of the first inner conductor 52 may be removed in order to provide the distance d over which the first inner conductor 52 extends starting from the T-junction 40.

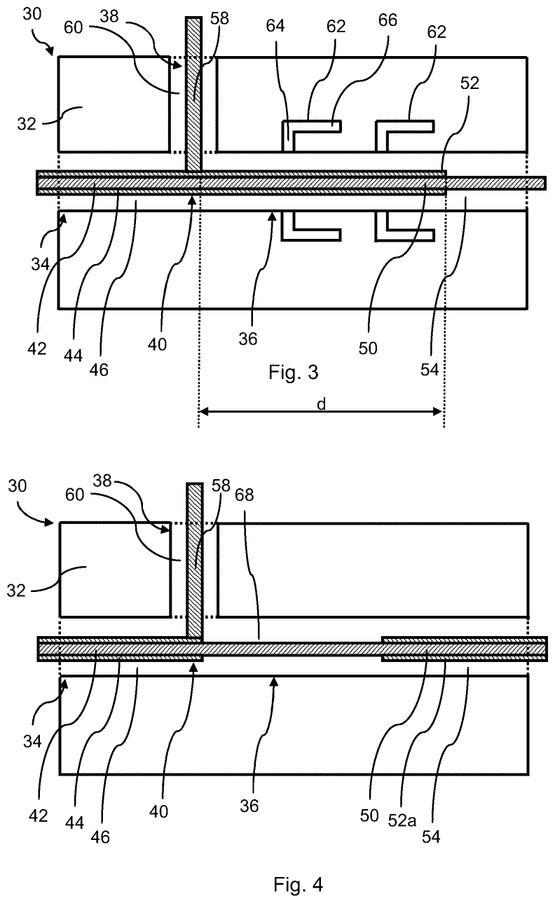

The combiner 30 of FIG. 3 corresponds to the combiner 30 of FIG. 2 except for the following differences.

The combiner 30 of FIG. 3 further includes one or more chokes 62 which are provided for stopping the propagation of radiofrequency and/or microwave frequency along the first transmission line section 36. Each choke 62 is a cavity within the body 32 filled with air and in fluid connection with the first outer conductor 56.

Each choke 62 has an L-shape in half cross-sectional view of the combiner 30. The choke 62 may be axially symmetrical to the first transmission line section 36. The choke 62 may include a first choke section 64 and a second choke section 66 which are both air-filled cavities in the body 32 and in fluid-communication with each other.

The first choke section 64 can protrude from the first transmission line section 36, optionally perpendicular to the first transmission line section 36. The first choke section 64 may be a disk-shaped cavity around the first transmission line section 36.

The second choke section 66 may extend parallel to the first transmission line section 36. The second choke section 66 can be formed as hollow cylinder, optionally coaxially arranged to the first transmission line section 36.

The body 32 of the embodiment of FIG. 3 is made from an electrically conductive material. In this case, it is possible that the combiner 30 does not include the common outer conductor 48, the first outer conductor 56, and the second outer conductor 61.

In order to reduce backscattering at the proximal end of the first inner conductor 52, the distance d may be chosen to be n*λ/2, wherein n is an integer equal to or greater than 0 and λ is the wavelength of the conveyed radiofrequency and/or microwave radiation in the first transmission line section 36.

The chokes 62 and the chosen length of the inner conductor 52 complement each other in reducing the transmission of radiation at two different frequency ranges. For example, the chokes 62 stop microwaves, and the chosen length of the first inner conductor 52 is designed to give smooth transition into the second transmission line section 38 at another frequency as well as stopping every lower frequency.

The combiner 30 of FIG. 4 corresponds to the combiner 30 of FIG. 3 except for the following differences.

The first transmission line section 36 does not include a first inner conductor 52. This corresponds to the situation in which the integer equal to 0 is chosen for the distance d. This means that the common inner conductor 44 ends at the T-junction 40.

The combiner additionally includes an additional first inner conductor 52a, but no first inner conductor 52. The additional first inner conductor 52a forms a gap 68 which extends between the T-junction 40 and the point where the additional first inner conductor 52a starts/ends. However, the gap 68 may be arranged at different positions of the first transmission line section 36. For example, the first inner conductor 52 ends at the distance d equal to n*λ/2 after the T-junction 40. The additional first inner conductor 52a is separated by the gap 68 of sufficient length such from the first inner conductor 52 such that there is no coupling of electrical signals over the gap 68.

The additional first inner conductor 52a can be omitted. In this case, the common inner conductor 44 ends at the T-junction 40 and no electrical connection exists with the first transmission line section 36. The first transmission line section 36 includes no inner conductor at all.

The combiner 30 of FIG. 4 does not include chokes 62.

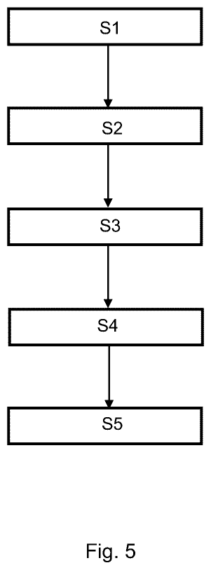

A method for manufacturing the cable assembly 10 is discussed in conjunction with the block diagram of FIG. 5.
In step S1, a coated optical fibre including the optical fibre 14 and the inner conductive layer 16 is provided. For example, the coated optical fibre may be purchased from manufacturers of coated optical fibres.
In optional step S2, the coated optical fibre may be coated with the coating layer 24. As described earlier, the coating layer 24 may include gold or silver. Any techniques for applying a layer of gold or silver to the inner conductive layer 16 may be used.
In step S3, the dielectric layer 18 is applied to the coated optical fibre. For example, die electric layer 18 is provided by co-extruding with materials such as expanded PTFE.
In step S4, the outer conductive layer 20 is applied to the dielectric layer 18. Commonly known techniques for applying an outer conductor to a dielectric layer 18 can be used.
In optional step S5, the cover layer 22 is applied to the outer conductive layer 20. To this end, commonly known techniques for applying a cover layer 22 may be used.

1. A cable assembly for an electrosurgical instrument, comprising
an inner conductive layer,
an outer conductive layer arranged coaxially with the inner conductive layer,
a dielectric layer separating the inner conductive layer and the outer conductive layer, and
an optical fibre for transmitting electromagnetic radiation in the ultraviolet spectrum, the visible spectrum, and/or in the infrared spectrum,
wherein the inner conductive layer, the dielectric layer, and the outer conductive layer form a coaxial cable providing a transmission line for conveying radiofrequency and/or microwave radiation,
wherein the inner conductive layer surrounds the optical fibre, and
wherein the inner conductive layer and the optical fibre are bonded to each other.

2. The cable assembly according to claim 1, wherein the inner conductive layer and the optical fibre are bonded to each other by a chemical bond, optionally by a molecular bond.

3. The cable assembly according to claim 1 or 2, wherein the optical fibre includes a core portion and a cladding layer which surrounds the core portion, wherein optionally the inner conductive layer and the cladding layer are bonded to each other.

4. The cable assembly according to claim 3, wherein the core portion has a diameter between 6 μm and 650 μm, the cladding layer has a diameter of between 80 μm and 700 μm, and/or the inner conductive layer has a diameter between 120 μm and 800 μm, optionally 350 μm.

5. The cable assembly according to any preceding claim, further comprising a coating layer surrounding the inner conductive layer.

6. The cable assembly according to claim 5, wherein the coating layer includes silver or gold, wherein optionally the silver coating layer has a thickness corresponding to the Skin depth of the radiofrequency and/or microwave radiation or wherein the gold coating layer has a thickness smaller than the Skin depth of the radiofrequency and/or microwave radiation.

7. The cable assembly according to any preceding claim, further comprising a combiner including a common transmission line section, a first transmission line section, and a second transmission line section,
wherein an input of the common transmission line section is in communication with outputs of the first transmission line section and the second transmission line section.

8. The cable assembly according to claim 7, wherein the common transmission line section includes a common optical fibre section connected to the optical fibre, a common inner conductor connected to the inner conductive layer, and optionally a common outer conductor connected to the outer conductive layer, and/or
the first transmission line section includes a first optical fibre section connected to the common optical fibre section and configured to be connected to a light source, and optionally a first outer conductor connected to the common outer conductor, and/or
the second transmission line section includes a second inner conductor connected to the common inner conductor and configured to be connected to an external radiofrequency and/or microwave energy source, a second dielectric portion electrically insulating the second inner conductor, and optionally a second outer conductor connected to the common outer conductor.

9. The cable assembly according to claim 8, wherein the combiner includes an electrically conductive body, wherein optionally the combiner is devoid of the common outer conductor, the first outer conductor, and/or the second outer conductor.

10. The cable assembly according to claim 8 or 9, wherein
the first transmission line section includes a first inner conductor connected to the common inner conductor, and
a length of the first inner conductor between an end of the first inner conductor and a junction of the second transmission line section and the common transmission line section is equal to n*λ/2, wherein n is an integer equal to or greater than 0 and λ is the wavelength of the conveyed radiofrequency and/or microwave radiation,
wherein optionally the first transmission line section further includes an additional first inner conductor which is arranged axially spaced apart from the first inner conductor.

11. The cable assembly according to claim 8 or 9, wherein the first transmission line section includes no first inner conductor and/or no additional first inner conductor.

12. The cable assembly according to any one of claims 8 to 11, wherein the common transmission line section includes a common dielectric portion and the first transmission line section includes a first dielectric portion arranged between the first optical fibre section and the first outer conductor, wherein the common dielectric portion, the first dielectric portion, and/or the second dielectric portion include solid polytetrafluoroethylene (PTFE), wherein optionally the dielectric layer includes expanded PTFE.

13. The cable assembly according to any one of claims 8 to 12, wherein the combiner includes at least one choke for preventing propagation of radiofrequency and/or microwave radiation, wherein the at least one choke is connected to the first transmission line section.

14. The cable assembly according to claim 13, when dependent on claim 9, wherein the choke includes a cavity within the electrically conductive body which is in fluid connection with the first outer conductor, wherein optionally, in a sectional view of the choke, a half of the choke has an L-shape.

15. A method for manufacturing a cable assembly, comprising the steps of
a) providing a coated optical fibre, wherein the coated optical fibre includes an optical fibre which is coated by a conductive layer, wherein the conductive layer is bonded to the optical fibre,
b) covering the coated optical fibre with a dielectric layer, and
c) covering the dielectric layer with an outer conductive layer.

16. The method according to claim 15, wherein the coated optical fibre is coated with a coating layer before step b), wherein the coating layer includes silver or gold.
